(19) United States
(12) Patent Application Publication
(10) Pub. No.: US 2023/0014648 A1
(43) Pub. Date: Jan. 19, 2023

(54) Determining Capsule Specificity for Specific Cell Types

(71) Applicants: CapCo Bio GmbH, Freiburg (DE); Albert-Ludwigs-Universität Freiburg, Freiburg (DE)

(72) Inventors: Irina Nazarenko, Glottertal (DE); Holger Klapproth, Freiburg (DE); Arnold Martin, Emmendingen (DE)

(21) Appl. No.: 17/786,678

(22) PCT Filed: Dec. 17, 2020
(86) PCT No.: PCT/EP2020/000212

§ 371 Date: Jun. 17, 2022

(30) Foreign Application Priority Data
Dec. 19, 2019 (EP) 19000577.7

(51) Int. Cl. A61K 9/51

(52) U.S. Cl. CPC A61K 9/5146; A61K 9/5115; A61K 9/5161; A61K 9/5169; A61K 9/5192

(57) ABSTRACT

The task of the invention is therefore making available transfer capsules that are taken up by the target cell type and permanently or transiently modify the target cell, without exerting any toxic effects on the cell during this process.

The solution according to the invention consists of the use of monodisperse cores, so as to produce polyelectrolyte nanocapsules having cell-specific sizes from them. The sizes for hematopoietic cells are in a range of 20-80 nm, preferably in a range of 40-60 nm. In this regard, the sizes of the particles must be in a very narrow range, so as to prevent toxic effects from occurring. In order to keep the toxicity of the nanocapsules low, it is furthermore important to remove the nanoparticles around which the capsules are built up (cores) before use. Methods in this regard are known from the state of the art (for example dissolution by means of EDTA).

A further task is the stabilization of the transfer capsules.

The solution according to the invention consists in the modification of the capsules, the layers and/or the cargo to be packed, by means of functional groups, which allows stabilization and thereby long-term storage at room temperature.

The third task is the targeted introduction of the transfer capsules.

The solution according to the invention is a functionalization of the layers by way of chemical modifications and/or supplementing of the layers with antibodies, proteins or peptides.

### CROSS-REFERENCE TO RELATED APPLICATIONS

This application is the United States national phase of International Application No. PCT/EP2020/000212 filed Dec. 17, 2020, and claims priority to European Patent Application No. 19000577.7 filed Dec. 19, 2019, the disclosures of which are hereby incorporated by reference in their entirety.

### BACKGROUND OF THE INVENTION

### Field of the Invention

Polyelectrolyte nanocapsules as a transport medium for biomolecules and “small molecular compounds” in cells are described in WO 002019020665 A1. These can be built up by means of what is called the layer by layer technique, around solid or liquid cores. In this regard, the size of the nanocapsules depends on the size of the cores used. For the selection and the efficiency of the uptake into specific cells, not only the surface structure but also the size of the nanoparticles is important. In WO 002019020665 A1 it is described that the capsules are formed around cores composed of calcium carbonate, wherein these cores are formed by means of a precipitation reaction, from a solution of calcium nitrate and sodium carbonate. Diverse aids influence the precipitation and ensure different sizes of the precipitated calcium carbonate cores.

### Description of Related Art

One problem in this regard is that there is a very large spread of the size distribution, i.e. in the production of particles of 60 nm, particles greater than 100 nm are also produced, and vice versa. However, it is absolutely necessary for successful use of these nanocapsules in the case of specific cell types that the size of the particles can be established to approximately 10 to max. 20 nm, so that uptake into undesired cells can be reduced. This is because hematopoietic cells can be transfected, above all, with nanocapsules having a size of 20-60 nm, and for tumor cells sizes of 100-200 nm are preferred. In other words, in order to avoid non-specific effects caused by non-specific uptake, the nanocapsules must have the right size.

A further significant problem in the state of the art consists of the toxicity of particles above 100 nm for cells having a hematopoietic origin, such as, for example, CD34+ hematopoietic precursor cells and T-cells. Until now, it has only been possible to transduce or electroporate such cells by means of the use of viruses. With all the related problems.

A further significant problem in the current state of the art is comparatively low stability of the capsules filled with cargo. Until now, only siRNA-charged capsules have proven to be extremely stable; other molecules, above all mRNA and DNA, are unstable in capsules and prevent long-term storage, which results in the degradation of the genomic cargo and/or in capsule aggregation. Furthermore, during long-term storage diffusion effects occur in the case of small molecules and chemotherapeutic medications, and this also does not allow effective use of these substance classes at the present point in time. Stability can be clearly improved by means of chemical modification of the capsule layer and/or of the RNA or DNA. Furthermore cell-specific or organ-specific introduction can be achieved by way of these modifications.

The particles produced using the bottom-up process until how have the problem that they have a broad size distribution and that the production process is very variable. The method is therefore not suitable for technical production of uniformly large nanoparticles. In contrast, particles produced using the top-down method can be obtained in a simple and inexpensive manner, but they have the problem of the non-uniform shape.

### SUMMARY OF THE INVENTION

The first task of the invention is therefore making available a capsule that is taken up by the target cell type and modifies the target cell permanently or transiently, without exerting any toxic effects on the specific target cell type in the process. The second task is stabilization of long-term storage by means of the chemical modifications mentioned above.

The capsules according to the invention consist of the use of mono-dispersed nanoparticles, so as to produce from them polyelectrolyte nanocapsules having cell-specific sizes, and, as needed, chemical modifications, wherein the sizes for hematopoietic cells are in a range of 20-80 nm, preferably in a range of 40-60 nm, for epithelial cells and non-hematopoietic cells of 60-280 nm, as described in WO0020199020665. In this regard, the sizes of the particles must be in a very narrow range, so as to prevent toxic effects from occurring. Biodegradable polymers, for example dextran and poly-L-arginine, which are used for building up the capsule, can be chemically modified as necessary (by means of linking functional groups, such as functional groups that contain hydrocarbon, oxygen, nitrogen, sulfur or phosphate; method for this are known from the state of the art), so as to prevent spontaneous aggregation and spontaneous cargo diffusion of the capsules and to improve the stability of the capsule. In order to keep the toxicity of the nanocapsules low, it is furthermore advantageous, in some cases, to remove the nanoparticles around which the capsules are built up (so-called cores) before use. Methods in this regard are known from the state of the art (for example dissolution by means of EDTA).

A sufficiently narrow size selection cannot be carried out efficiently using the cores obtained by means of precipitation, since the distribution in size is very broad. Within the scope of this invention, nanoparticles and nanocapsules having a narrowly defined size distribution of ±15 nm or optionally ±25% around the median value, preferably ±10 nm (or ±20% around the median value) are referred to as monodisperse.

Surprisingly, the problem of the size of the cores can be solved not only by means of the use of size-sorted, mechanically produced nanoparticles. Thus, nanoparticles having a size of 15-60 nm can be produced by means of grinding calcium carbonate in ball mills, and then uniform fractions (±10 nm) can be produced by means of fractionation (by means of sedimentation or centrifugation). These particles can then be coated with polymers using the layer by layer method. However, the non-uniform shape of these particles is disruptive, since it disturbs sorting of the particles. Only further purification of these particles, for example by means of cross-flow filtration after dissolution of the cores, allows a sufficiently precise size selection.

Nanocapsules can be produced by means of the known layer by layer method. Molecules such as chemotherapeutic medications, small molecules, and macromolecules such as nucleic acids and proteins, can be introduced between the layer, and these can then be released into the cell. For example, chimeric T-cell receptors can be produced by means of introducing nanocapsules charged with DNA into T-cells. RNA molecules can also be introduced into cells, so as to produce an only transient modification of the cells. Furthermore, all the aforementioned molecules and macromolecules can be introduced either individually or in any conceivable combination. This property allows transfection of CRISPR/Cas complexes, for example. The aforementioned chemical modifications can be used both for the capsule polymers and for the cargo, so as to regulate stability and consequently release in the target cells.

Primary cells are cells that were taken from the body and have not lose their tissue-specific properties due to cultivation. Primary cells are, for example but not exclusively, stem cells, reproductive cells, hematopoietic cells, tumor cells or also mesenchymal stem cells, tissue cells in vivo and ex vivo.

Body cells that can differentiate into different cell types or tissue are referred to in general as stem cells. Depending on the type of stem cell and how it is influenced, they have the potential of developing into any kind of tissue (embryonic stem cells) or into specifically established tissue types (adult stem cells/precursors).

Suitable polymers for building up layers of the nanocapsules are polycations and polyanions, which alternately form the layers of the nanocapsules. Due to the opposite charge, the polymers can form the layers by means of self-organization. After formation of each layer, the part of the polymers that have not been must be separated from the nanocapsules. Polymers in the sense of the invention comprise not only traditional polymers and copolymers but also biopolymers such as macromolecules having an orderly structure composed of amino acids, nucleic acids, and polysaccharides or oligosaccharides.

Examples of polycations are:
Polyethylenimine (PEI),
Chitosan (CS),
Poly(L-lysine) (PLL),
Poly(amido amine)s (PAA),
Oligolysines,
Poly-(I)-ornithines (PLO),
(Poly(diallyldimethylammonium chloride) (PDAC),
Histidines and lysine-rich peptides and proteins, for example Cys-Gly-Ala-Gly-Pro-(Lys)3-Arg-Lys-Val-Cys.

Examples of polyanions are:
Poly(methacrylic acid) (PMA),
Poly(N-vinylpyrrolidone) (PVP),
Tannic acid (TA),
Poly(2-n-propyl-2-oxazoline)s (PnPropOx),
Poly(l-vinylimidazole),
Poly(lactic-co-glycolic acid) (PLGA),
Dextran sulfate,
Polyethylene glycol,
Bovine serum albumin,
Human serum albumin,
Protamine sulfate,
1-ethyl-3-(3-dimethylaminopropyl) carbodiimide (EDC).

Substances that can be used for stabilization of the nucleic acids are substances that are applied in the capsules together with the nucleic acids or substances that diffuse in the capsules by means of incubation with the finished capsules, and diffuse into the lumen of the capsules and remain there, at least in part, until entry of the capsules into the cells. Examples of such substances are:
Protamine sulfate,
Cell penetrating peptide,
Glutathione,
TAT protein,
RNAse inhibitors,
DNAse inhibitors.

Examples of nanoparticles that are suitable of cores for the production of nanocapsules:
Iron nanoparticles (including magnetite),
Silica nanoparticles,
Commercially available CaCO₃ nanoparticles
Other carbonate nanoparticles such as MgCO₃,
Vesicles,
Gold nanoparticles,
Latex nanoparticles.

The surface of the nanoparticles can be modified by means of further groups, so as to improve the penetration of the target cells. Such groups are, for example, folic acid groups, COOH groups, NH₃ groups. These groups can also be used for bioconjugation, so as to bind specific binding molecules such as ligands and receptors, including antibodies, to them.

### EXAMPLE 1: FRACTIONATION OF CALCIUM CARBONATE NANOPARTICLES

Calcium carbonate nanoparticles were procured from SkySpring Nanomaterials. The average particle size was 15-40 nm. Using the particles, a 10 mg/ml suspension in PBS was produced. Ultrasound was used for 5 minutes so as to separate the particles. They were then purified by size by means of fractionated centrifugation in an Eppendorf centrifuge. 2 ml suspension were centrifuged at 500 RCF, 1000 RCF, 2000 RCF, 5000 RFC [sic], 10,000 RCF, 15,000 RFC and 20,000 RCF. This process started at the lowest RCF, the top fraction was removed, and centrifugation continued at the next higher RCF. The pellet of the centrifugation was subsequently measured. The 500 RCF fraction was discarded (particles were aggregated or too large).

### EXAMPLE 2: PRODUCTION OF THE POLYELECTROLYTE NANOCAPSULES

In order to coat the nanoparticles, dextran sulfate (as a sodium salt) and poly-L-arginine hydrochloride were used as described in WO 002019020665 A1. During this process, it is possible to introduce one or more nucleic acids (plus additional biomolecules) between the layers. The nucleic acids adhere to the capsule wall by means of electrostatic binding, so that incubation of the capsules with the biomolecules is sufficient. The method is described in WO 002019020665 A1.

It was possible to modify these chemically, so as to change the stability during storage and the decomposition time in the cell. Chemical modifications by means of the use of one or more functional groups, such as hydrocarbons, groups that contain oxygen or nitrogen, groups that contain sulfur (S/SH), N/NH, and other groups that contain P were introduced and tested. The chemical modifications showed, in preliminary experiments, that it was possible to guarantee storage for a longer period of time. Furthermore, it was not possible to store the capsules at room temperature for a time interval, as well, and this represents a significant improvement for transport (freight), in particular, since it will lower costs and administrative effort (key word: customs regulations involving dry ice and refrigerated products).

### EXAMPLE 3: PURIFICATION OF THE FINISHED NANOCAPSULES BY MEANS OF TANGENTIAL FLOW FILTRATION

Finished nanocapsules were then purified to remove larger nanocapsules that might be present, using a KrosFlo Research Ili System with a 50 nm filter module. Particles having a size of more than 50 nm were retained. For this purpose, 50 ml of a 10 mg/ml suspension of the nanocapsules in PBS was produced. This was filtered according to the manufacturer's instructions.

### EXAMPLE 4: SEPARATION AFTER ASYMMETRICAL FLOW FIELD FRACTIONATION (AF4)

Finished nanocapsules were then purified to remove larger nanocapsules that might be present, using an Eclipse AF4 from Wyatt Technology. In this process, particles were separated according to charge and size. For this purpose, 50 ml of a 10 mg/ml suspension of the nanocapsules in PBS was produced. This was separated according to the manufacturer's instructions.

### EXAMPLE 5: TRANSFECTION OF PRIMARY CELLS

For hematopoietic cells, above all, nanocapsules having a size of 40-80 nm are clearly more advantageous. Protein, DNA, mRNA, miRNA and siRNA were used as cargo for nanocapsules having a size of 50 nm to 80 nm. CD 34+ hematopoietic stem cells, CD4+ and CD8+ T cells were incubated with the capsules for 48 hours. In this process, 10 capsules/cell were used. Successful introduction was monitored by means of confocal microscopy in the case of the fluorescence-marked capsules and PCR.

### EXAMPLE 6: TRANSFECTION OF FURTHER PRIMARY CELLS

For embryonic stem cells as well as for induced pluripotent stem cells (iPS-cells), nanocapsules having a size of 50-120 nm were produced. Protein, DNA, mRNA, miRNA and siRNA were used as cargo for nanocapsules having a size of 50 to 120 nm. Embryonic stem cells and iPS-cells were incubated with the capsules for 48 hours. In this process, 20 capsules/cell were used. Successful introduction was monitored by means of confocal microscopy in the case of the fluorescence-marked capsules and PCR.

### EXAMPLE 7: STABILIZATION AND STORAGE AT ROOM TEMPERATURE (RT) OF CAPSULES

For all the capsules listed in the above examples, further modifications of the cores or of the layers were carried out by means of introducing functional groups such as hydrocarbons, groups that contain oxygen, nitrogen, groups that contain sulfur (S/SH), N/NH, and other groups that contain P, and thereby it was possible to achieve stabilization of the capsules and thus the desired goal of storage at RT.

### EXAMPLE 8: INFLUENCING THE NATURAL DECOMPOSITION OF THE CAPSULES

By way of the modifications introduced in Example 2 and 7, of functional groups on the core as well as on the layers, it was possible to influence the natural decomposition of the capsules after introduction into the corresponding target cell. This resulted in decomposition times per layer at 4 hours in mesenchymal and tumor cell lines, for example, as well as decomposition times of 24 hours in cells having a hematopoietic origin.

### EXAMPLE 9: TARGETED INTRODUCTION: USING ANTIBODIES, PEPTIDES, PROTEINS

For further specification of the targeted introduction of cargo into desired target cells, corresponding antibodies, peptides or proteins were introduced into the outermost layer of the capsules.

### EXAMPLE 10: STABILIZATION OF CAPSULES HAVING SMALL MOLECULES AS THE CARGO

In order to stabilize the capsules that are charged with small molecules or chemotherapeutic medications, the small molecules or chemotherapeutic medications were immobilized in the capsules by means of click chemistry, and thereby diffusion effects were reduced.

1. A nanocapsule suitable for introduction of target molecules of primary cells and stem cells, wherein
the capsules consist of at least two biodegradable layers,
between these at least two layers, at least one nucleic acid, protein and/or small molecule or other target molecule is situated,
the nanoparticle cores have a firmly defined size with a variance of maximally 25%,
likewise the nanoparticles/cores can be separated by charge in addition to their size,
as needed, the capsule surface and/or individual layers within the capsule can be modified using functional groups.
likewise cargo elements between the layers or the layers themselves can be modified.

2. The nanoparticle cores according to claim 1, wherein the nanoparticles having a fixed size are selected from the size range of 20-80 nm.

3. The nanoparticle cores according to claim 2, wherein the nanoparticles are selected from the size range of 30-60 nm.

4. A method for introducing cargo into primary cells or cell lines, wherein the nanocapsules are brought into contact with cells, wherein
the nanoparticle cores have fixed, defined size with a variance of maximally 25%,
the nanocapsules consist of at least two biodegradable layers,
between these at least two layers, at least one nucleic acid, protein, small molecule and/or chemotherapeutic medications is/are situated,
one or more layers or cargo can be modified by means of binding of the functional groups, so as to regulate the stability during storage or the degradation time in the cells.

5. The nanocapsules according to claim 1, modified by means of functional groups for stabilization purposes.

6. The nanocapsules according to claim 1, for targeted transfer.

7. The method according to claim 5, wherein the cells are healthy or malignant cells having a hematopoietic origin.

8. The method according to claim 6, wherein the cells are transiently transfected.

9. The method according to claim 6, wherein the cells are transfected in a stable manner.

10. The method according to claim 6, wherein the cells are embryonic cells or iPS cells.

11. A method for producing nanocapsules, wherein the cores for building up the nanocapsules are monodisperse nanoparticles, and that the nanocapsules are charged with nucleic acids, proteins, small molecules or chemotherapeutic medications, and are used modified with functional groups or not modified.
